(19) United States
(12) Patent Application Publication
(10) Pub. No.: US 2004/0176821 A1
(43) Pub. Date: Sep. 9, 2004

(54) Vision rehabilitation method and device

(76) Inventors: Jean Delbeke, Mouscron (BE); Benoit Gerard, Chaumont-Gistoux (BE); Claud Veraart, Bruxelles (BE)

Correspondence Address: William M Lee, Barnes & Thornburg, PO Box 2786, Chicago, IL, 60690-2786, US

(21) Appl. No.: 10/476,240

(22) PCT Filed: May 3, 2002
(86) PCT No.: PCT/EP02/04956

Related U.S. Application Data
(60) Provisional application No. 60/288,583, filed on May 3, 2001.

(30) Foreign Application Priority Data
Jan. 21, 2002 (GB) 0201182.3

(51) Int. Cl. A61N 1/18

(52) U.S. Cl. 607/54

(57) ABSTRACT

The present invention relates to a method and a device for visualising an image by electrical stimulation of functional parts of a visual system. A method according to the present invention comprises an image provision step for providing an image to be visualised by the visual system, a selection step for selecting, from a phosphene data file comprising a description of phosphenes and the stimuli characteristics needed for obtaining them, a number of most appropriate phosphenes to be generated for visualising the image, so that the image is transferred into a summation of patches of light, and a stimulation step for stimulating functional parts of the visual system so as to generate the selected phosphenes. This way, a pixel based image is converted into a plurality of patches of light. A device for carrying out the method is also described.

### TECHNICAL FIELD OF THE INVENTION

The present invention relates to the field of visual prosthetic devices for blind or poorly sighted people, more specifically to a method and a device for generating signals, the signals being suitable for visualising an image by electrical stimulation of functional parts of a visual system, e.g. of a human.

### BACKGROUND OF THE INVENTION

Three different approaches to an implantable visual prosthesis can be distinguished on the basis of a functional part of the visual system being electrically stimulated: the occipital cortex, the retina, or the optic nerve.

In the field of visual prosthetic devices, it is known that electrical stimulations characterised by parameters such as shape, size, number and frequency of the pulses of electrical stimulation can generate a local visual perception, called a phosphene. Blind people can thus obtain a rehabilitated vision whereby images from an image grabbing device such as a TV camera, translated into a set of stimulations carried to the visual system, can generate a number of phosphenes appropriately timed and located to be seen as similar or efficiently representing the captured image.

It is for example known from Wm. H. Dobelle, “Artificial vision for the blind by connecting a television camera to the visual cortex”, ASAIO Journal 2000 Jan-Feb; 46(1): 3-9, to provide a visual prosthesis by connecting a digital video camera, a computer and associated electronics to electrodes connected to the visual cortex of the brain of a blind person. When stimulated, each electrode produces closely spaced phosphenes. The phosphene map differs from ones reported by other researchers and consisted of point phosphenes. The patient had tunnel vision—the area of vision was estimated as 8 by 3 inches at arm's length. A clear description of the method of converting pixels into phosphene stimulation is not provided. Where the resolution of the captured image exceeded that of the phosphene map, pixels were aggregated, e.g. 2, 4, 8, 16 pixels were combined to create a single pixel for transmission to the patient. The image is provided to the patient in frames—each implanted electrode being stimulated by a train of six pulses at 30 Hz to produce the frame. Due to the close spacing of the phosphenes a pixel of the image can be associated with the closest phosphene.

In U.S. Pat. No. 5,935,155 is described a visual prosthesis comprising a camera for perceiving a visual image and generating a visual signal output, retinal tissue stimulation circuitry adapted to be operatively attached to the user's retina, and wireless communication circuitry for transmitting the visual signal output of the camera to the intra-ocular retinal tissue stimulation circuitry. The visual signal output is used to generate stimulation current signals to be used by an electrode array of the retinal tissue stimulation circuitry which stimulates the retinal cells to produce phosphenes in a pattern to simulate vision.

In the above methods and devices, it is difficult to efficiently translate an instantaneous image into the most appropriate electrical stimulation of suitable parts of the visual system in order to faithfully carry over the related visual information. Up to now, the problem set forth above is implicitly solved using a point-to-point method whereby supposedly point-like phosphenes are generated according to the luminosity of corresponding image pixels. Although straightforward, this method has several drawbacks.

The first is that, in case of stabilised images, continuous stimulation of the same retinotopic part of the visual system leads to extinction of the perception through a well-known physiological phenomenon called ‘fixed image effect’. Normal sighted people do not suffer from it because of continuous small movements of the eye with automatic position compensation in the brain.

Another difficulty requiring solution is the fact that in at least some patients and with at least stimulation methods the phosphenes generated extend beyond the size of a single pixel they are supposed to represent.

Furthermore, as most visual prosthesis studies have shown, the set of phosphenes that can be generated in a given subject is limited and should be defined by preliminary testing. A suitable procedure is thus required to fit any prosthetic system with this kind of personal data about available phosphenes.

Finally, in “Visual sensations produced by optic nerve stimulation using an implanted self-sizing spiral cuff electrode”, C. Veraart et al., Brain Research 813 (1998), pp.181-186 and in WO 00/06248 is described an electrode implanted around the optic nerve of a totally blind person. Electrical stimuli applied to the nerve produce localised visual sensations (phosphenes) broadly distributed throughout the visual field. These visual sensations can be varied by changing the stimulation conditions (pulse duration, pulse train frequency, pulse intensity). There is no question of connecting each point or pixel of the image to be visualised to one of the optic nerve fibres, as the optic nerve contains about a million distinct fibres. The only possibility to visualise the image is to selectively send signals to certain bundles of fibres by an electrode with e.g. four contacts.

The state-of-the-art in the field of the proposed invention, relates to the encoding of picture elements grabbed by a device such as a TV camera, into appropriate electrical stimulation conditions of the visual system, in order to elicit a perception corresponding essentially to the visual information contents of the input image. Such encoding usually relates to image processing. It appears that most publications or patents in the field of image encoding, such as U.S. Pat. No. 4,222,076, relate to technical improvements of image quality using image decomposition. The present invention, however, is only dealing with eliciting a perception roughly resembling to the input image.

Existing systems are limited to the use of straightforward pixel-to-point cortex or retina stimulation. Indeed, the existing literature in the field implicitly refers to a code based on a matrix of supposedly ideal punctual phosphenes corresponding to a matrix of pixels. However, it is not obvious how to convert the phosphenes, which are light impressions as uncoordinated flashes or dots, into an image that is useful.

### SUMMARY OF THE INVENTION

It is an aim of the present invention to improve or to optimise the rehabilitation of vision in a blind or poorly sighted person, more particularly by the use of phosphenes over a portion of the visual field, preferably making use of only a few contacts and a few stimulators. The phosphenes are generated by stimulating a functional part of the visual system, such as the visual cortex, the retina, or the optic nerve. It is therefore an object of the present invention to provide apparatus and methods for improving or optimising the rehabilitation of vision of poorly sighted person.

This object is solved in accordance with the present invention by provision of methods and apparatus for transformation of a pixel-based image to a phosphene-based image in such a way that the location and/or size and/or shape of the phosphenes is used in optimising the representation of the image. In particular the position of a phosphene as visualised can be moved by the generation of suitable electrical signals.

The present invention may provide a method and a device that uses the output signal of an image-grabbing device such as e.g. a video camera, to provide an image. In accordance with one embodiment, this output signal is converted in a digital format using an analog-to-digital converter, and the obtained digital images are preferably pre-processed. Pre-processing may include, for example, resolution adaptation, thresholding, spatial and temporal filtering, selective spatial decimation (through a mapping table), contrast enhancement, segmentation into regions, edge detection and gain modulation by local movement. An additional global movement detection can also be used. From here on, the word ‘pixel’ in the present patent application will refer to the elements of such an optionally pre-processed image to be visualised. The image is normally represented in an array, e.g. a Cartesian or log-polar array or pixels.

A method of the present invention requires personal data about available phosphenes in a person. Phosphene data is provided preferably as a single digital phosphene data file or as a set of digital phosphene data files such as look-up tables or sets of parameter equations or a combination of these. These phosphene data files preferably contain records of all phosphenes which can be used or a sub-set thereof. The location, size and shape and optionally the orientation of each phosphene is preferably recorded. Other phosphene attributes may also be recorded, e.g. intensity, texture, colour. Preferably, all possible synchronous stimulation sets are included whether they involve single electrode contact or multi-electrode contact stimulations. Besides the stimulus characteristics most likely to generate a given phosphene, each record in these phosphene data files contains, for each stimulus characteristic, a representation of the generated phosphene and preferably a description of its attributes, among which can be at least one of its brightness value, colour, structure, texture, orientation, size, shape. In one embodiment of the present invention, this representation and description can be limited to a central position and a size factor of the phosphene, e.g. a centre point and a radius or representative distance. Optionally, an orientation may also be included as well as two dimensions for non-symmetrical phosphenes, e.g. a major and minor axis. Alternatively, a contour of a phosphene may be recorded or the coefficients of an equation which defines the shape of the phosphene, e.g. by its contour, for instance, an ellipse or circle.

A method to obtain the typical localisation or position and characteristics of a phosphene is provided, whereby stimulus strength (defined as a ratio of stimulation current to threshold or as a relative difference between a stimulation current and the threshold, the threshold being the level at which a transition occurs from a state of no perception to a state of seeing a phosphene) is used as a parameter, as well as the effects of current intensity, pulse duration, number of pulses in a stimulation signal and frequency of the pulses in the stimulation signal or pulse train. Model equations or mathematical functions are fitted to data obtained by tests performed on a person using a device according to the present invention. These mathematical functions may relate stimulus strength to pulse duration, number of pulses in a stimulation signal and frequency of the pulses in the stimulation signal in order to obtain phosphene characteristics. A table of possible phosphene positions can be established as follows: the frequency and number of pulses in a stimulation pulse train can be used to calculate a maximal eccentricity range available. The current intensity or pulse duration can be adjusted to locate the phosphene at an appropriate spot along the path between the maximal eccentricity and a central convergence point. Conversely, while making sure that the number of pulses and the frequency of the stimulation signal are such that the stimulation pulse duration is short enough to make the required stimulation frequency possible (i.e. e.g. 40 ms for a 25 Hz or 20 ms for a 50 Hz stimulation), a stimulus strength giving a desired phosphene position can be calculated. The current intensity and pulse duration can then be adjusted to obtain the required strength while minimising the duration and thus the stimulation charge while keeping it long enough to avoid saturation of stimulator circuitry of the device.

Similarly, different equations can be used to obtain the required phosphene area or brightness in function of the stimulus strength. If a given phosphene size is required, then the corresponding stimulus strength may be calculated and the number of pulses or frequency of the stimulation signal will be adjusted in such a way that the required position is still obtained.

The advantages of this method are that not all possible phosphenes to be used must be defined experimentally, as interpolation is possible with the established models or mathematical functions.

For some applications, tables can be replaced by equations which can be calculated on-the-fly, thus reducing the size of the required memory device.

Phosphene selection can be optimised for parameters such as the quantity of electric charge delivered or the total stimulus duration or the required voltage.

A method for generating stimulation signals suitable for visualising an image by electrical stimulation of functional parts of a visual system of an animal, according to an aspect of the present invention comprises: a phosphene data file providing step for providing a record of a location of each phosphene of a plurality of phosphenes, at least one phosphene having a different location than other phosphenes, a selection step for selecting out of the phosphene data file a phosphene for visualising a region of the image; and a stimulation signal generating step for generating stimulation signals for stimulating functional parts of the visual system so as to generate the selected phosphenes, at least one stimulation signal being suitable for changing the location of a phosphene in the visual system. The phosphene data file may also include other data such as a size parameter of each phosphene. The stimulation signal generating step may be such that at least some stimulation signals are suitable for changing the location of phosphenes so as to form an array. The array formed may be a regular array.

A method for generating stimulation signals for visualising an image by electrical stimulation of functional parts of a visual system of an animal, e.g. a human, according to another aspect of the present invention comprises: a phosphene data file providing step for providing a record of a location and a size parameter of each phosphene of a plurality of phosphenes, at least one phosphene having a different size than other phosphenes, a selection step for selecting out of the phosphene data file a phosphene for visualising a region of the image; and a stimulation signal generating step for generating stimulation signals for stimulating functional parts of the visual system so as to generate the selected phosphenes, the generating step using at least a size parameter of a selected phosphene. The size parameter may be an average radius, for instance. The phosphene data file may include other parameters of the phosphene such as brightness value, colour, structure, texture, orientation, area, size, shape. Any of these parameters may also be used in the stimulation signal generating step. The selection step involves selection of the most appropriate phosphene for a particular region of the image to be visualised. One criterion is to match a size parameter and a location of the phosphene to the size and location of the region of the image to be visualised. The region to be visualised may be one or a plurality of contiguous pixels of the image. ‘Most appropriate’ stands here for different criteria that can be applied in different embodiments of the system. In one embodiment the available phosphenes may be fitted to pixels of the image having the largest brightness. Phosphene size and orientation can also be taken into account. In other embodiments, the selection step may be based on fitting the whole phosphene area to the internal image. In other embodiments, phosphene shaping corresponding to image edges may be included in the decision process of which phosphenes are to be selected as being most appropriate.

According to another aspect of the present invention, an image provided in the form of pixels, may be converted into regions or patches of light by generating the most appropriate phosphenes. To achieve this the image to be visualised may be pre-processed into regions of similar light intensity and/or colour, e.g. by region segmentation (“painting-by-numbers” algorithms). Available phosphenes are then mapped to these regions at least based on the size and location of the available phosphenes.

The present invention may provide a method to detect coincidences between pixels of a pre-processed image, and available phosphene centres within the remaining visual field of the visually handicapped user. Due to the disease, it may be possible that not all parts of the visual field can be considered for phosphene production. A rehabilitation method according to the present invention may identify that remaining part of the visual field within which phosphene centre positions are to be located. This can be done by extracting, from the set of available phosphenes, those of which the centre lies at almost regular intervals along the remaining part of the visual field in order to cover it substantially entirely and in a substantially regular pattern. Such phosphenes may be calculated form mathematical functions relating stimulus strength to pulse duration, number of pulses in a stimulation signal and frequency of the pulses in the stimulation signal in order to obtain phosphene characteristics. Alternatively, stimulation parameters susceptible to generate phosphenes with centres regularly spaced along the remaining visual field, can be selected using a model of phosphene generation or mathematical functions relating different stimulation parameters to phosphene characteristics.

Another possibility is to use a table of stimulation parameters susceptible to generate phosphenes with centres spaced along the remaining visual field, for example regularly spaced. This can be done by changing the stimulation signal so as to move at least one phosphene in the visual system so as to form an array, e.g. a regular array. The regularly spaced phosphene centres are preferably spaced with a resolution similar, or identical, to the one of the pixels of the pre-processed input image, in order to build a grid susceptible to be superimposed to the pixels of the pre-processed input image. Given such a grid of regularly spaced phosphene centres, it is convenient to detect real-time coincidences between part of the input image pixels and grid holes, e.g. in every video frame. Coincidences can trigger phosphene generation using the stimulation parameters linked to the selected grid holes. Selection of adequate phosphene generation parameters among several grid holes coinciding with input image pixels can be done according to various rules. One can prefer to make this selection randomly, or on the basis of pixel brightness, or on the basis of location with respect to the remaining visual field centre, or other peculiar regions. Selection of phosphene generation parameters can also be done in view of previously selected phosphene generation parameters. Possible rules of selection based on previously selected parameters can be to select the least often selected grid hole, or the one located farthest away from the previous one, or a neighbouring one. Selection of phosphene generation parameters can also be adaptive, depending of the users strategy in certain behavioural conditions such as scanning of the environment with the camera-gaze; fixation of camera-gaze on an object, etc.

In accordance with an embodiment of the present invention, when several phosphenes are available, the phosphene or phosphenes not used for the longest period of time is/are selected. This can be implemented, for example, either by changing the start address of the search on each new search access to the phosphene data file, or by time labelling phosphene data in the phosphene data file(s) when each phosphene was last used. Another aspect of the present invention is to organise the structure of the phosphene data file in a specific order. The phosphene data file(s) are preferably arranged in such a way that the most efficient stimuli (i.e. covering the largest pixel groups), for example synchronous stimuli (single pulses or trains), can be used first. A slightly different embodiment of the phosphene data file(s) would involve splitting a single look-up table into, on the one hand, a phosphene position table and, on the other hand, a phosphene synonyms table from which the most appropriate equivalent can be selected after the right phosphene position is selected.

According to a preferred embodiment, synchronous stimulations, interleaved pulse trains and/or successive stimuli may be used in the stimulation signal generation step in order to optimise information transmission to the visual system. More particularly, persisting pixels or sets of pixels in the image to be visualised may be translated into successive stimulations. According to another embodiment, fixed image extinction is reduced through the selection of a succession of slightly different phosphenes to represent a same constant bright pixel in the image to be visualised.

A negative feedback step may be provided, whereby image data of the visualised image, corresponding to the generated phosphenes, is subtracted pixel by pixel from the provided image, and this compound image is then used as a provided image in a next selection step. That way, the whole system behaves as a low-pass filter in time.

In accordance with another aspect of the present invention, a device for generating stimulation signals suitable for visualising an image by electrical stimulation of functional parts of a visual system of an animal is also provided. The device comprises means for receiving an image to be visualised by the visual system, a phosphene data store for storing at least a size parameter and a location of each phosphene of a plurality of phosphenes, at least one phosphene having a different size than other phosphenes, a selector for selecting out of the phosphene data store a phosphene for visualising a region of the image, the selector using the size parameter of a selected phosphene, and a signal generator for generating the stimulation signals for stimulating the functional parts of the visual system so as to generate the selected phosphenes.

In accordance with another aspect of the present invention, a device for generating stimulation signals suitable for visualising an image by electrical stimulation of functional parts of a visual system of an animal is also provided. The device comprises means for receiving an image to be visualised by the visual system, a phosphene data store for storing at least a location of each phosphene of a plurality of phosphenes, at least one phosphene having a different location in the visual system than other phosphenes. At least one stimulation signal may be suitable for changing the location of a phosphene in the visual system. Therefore, the signal generator may comprise means for generating at least some stimulation signals suitable for changing the location of phosphenes so as to form an array, preferably a regular array.

The means for providing an image may comprise an image grabbing device for capturing an image, such as e.g. a camera.

Preferably, the selector comprises a look-up table comprising references to phosphenes, their size and location, and their corresponding stimulation parameters to be used by the signal generator to assemble all the stimulation signals required for an image. The selector may also comprise calculation means to calculate parameter equations describing the phosphenes, e.g. when the shape of the phosphene is described by an equation.

The signal generator may contain all necessary functions to autonomously deliver appropriate signals, e.g. voltages and/or electrical currents, to electrode contacts to be located in the visual system, e.g. implanted in the brain, attached to the optic nerve, etc. after having received stimulation parameters corresponding to the phosphenes selected by the selector.

According to a preferred embodiment, the signal generator comprises means for applying synchronous stimulations, interleaved pulse trains and/or successive stimuli to the functional parts of the visual system.

The device according an embodiment of the present invention preferably also comprises negative feedback means for feeding back to the selector visualised image data corresponding to the generated phosphenes. The luminance of each input pixel may be fed to a leaky integrator. All integrator outputs are sent to a selection unit where the brightest pixel or pixel group is selected. The phosphene data store is then examined to find the most appropriate phosphene or phosphenes to display this pixel or pixel group. The image that would be generated by this phosphene or these phosphenes is then fed back pixel-by-pixel to the input of the integrator as negative feedback, i.e. subtracted from the image stored in the integrator. The remaining image is then subjected to the same procedure, that is identification of the brightest pixel group, selection of the most appropriate phosphene or phosphenes, feed back of the image which would be generated by this phosphene or phosphenes, to the input of the integrator, etc. This procedure is repeated until the complete image is analysed. New images are added to an intensity-diminished version of the current image in the integrator so that previous images “leak” away.

In another aspect of the present invention a method for generating stimulation signals for visualising an image by electrical stimulation of functional parts of a visual system of an animal is provided, comprising the steps of:

an image receiving step for providing a first image to be visualised

a phosphene data file providing step for providing a record of a location of each phosphene of a plurality of phosphenes,

a selection step for selecting out of the phosphene data file a phosphene for visualising a region of the first image,

subtracting an image corresponding to the selected phosphene from the first image to form a second image, and

a stimulation signal generating step for generating stimulation signals for stimulating functional parts of the visual system so as to generate the selected phosphene(s).

In yet another aspect of the present invention a device for generating stimulation signals for visualising an image by electrical stimulation of functional parts of a visual system of an animal is provided comprising:

means for receiving a first image to be visualised,

a phosphene data store for storing at least a location of each phosphene of a plurality of phosphenes,

a selector for selecting out of the phosphene data store a phosphene for visualising a region of the first image,

means for subtracting an image of the selected phosphene from the first image to form a second image, and

a stimulation signal generator for generating stimulation signals for stimulating the functional parts of the visual system so as to generate the selected phosphene(s).

The present invention also includes the use of any of the above apparatus or methods of the invention with a suitable electrode or electrode array for providing a visualisation rehabilitation device or method. The present invention also includes such a device as implemented on an animal, especially a human. The present invention also includes a method of treatment of a person or animal suffering from blindness or impaired vision by implanting a suitable electrode or electrode array on or in the visual system of the person or animal and driving the electrode or electrode array with signals generated in accordance with the above methods or supplied by the above apparatus in accordance with the invention. Transfer of the signals from the signal generator to the electrode or electrode array can be made by any suitable method, e.g. cable or wireless transmission.

The present invention further provides a computer program product having code segments which provide the functionality of any of the methods according to the present invention when executed on a computing device associated with a device for generating stimulation signals suitable for visualising an image by electrical stimulation of functional parts of a visual system of an animal. The computing device may include one of a microprocessor and an FPGA, for example. Further, the present invention includes a data carrier such as a CD-ROM or a diskette which stores the computer product in a machine readable form and which executes at least one of the methods of the invention when executed on a computing device.

The system of the present invention has the advantage that it is largely customisable through the interchangeable character of the phosphene data files. Also, parameters such as image processing coefficients, integrator reset threshold on movements, mapping table, synonyms, integrator leak time constant, and selection unit parameters, are easily uploaded from an external computer if necessary. It maximises the information transmission with the means (phosphenes) available, applying synchronous, interlaced and alternate stimulation principles, with appropriate delays and image repetition rate. The individual phosphene spatial extension is taken into account to build a perceptive image. Simple adaptation of the data files can take into account some differences in phosphene luminosity as well as non-linear and time effects of individual phosphenes.

Other features and advantages of the present invention will become apparent from the following detailed description, taken in conjunction with the accompanying drawings, which illustrate, by way of example, the principles of the invention.

### BRIEF DESCRIPTION OF THE DRAWINGS

FIG. 1 is a graphic representation of the retinotopic arrangement of phosphenes generated by electrically stimulating a cuff electrode which encircles the optic nerve.

FIG. 2 is a schematic overview of a method according to the present invention.

FIG. 3 is a flow diagram of one embodiment of the present invention.

FIGS. 4A, 4B, 4C and 4D illustrate visual perception threshold Iᵥ in function of different stimulus parameters. FIG. 4A is a graph of Iᵥ in function of stimulus duration if a single pulse is used. FIG. 4B is a graph of Iᵥ in function of stimulus duration if 9 pulse trains at 80 Hz are used. FIG. 4C is a graph of Iᵥ in function of the pulse train frequency, for pulse trains of 9 pulses with a duration of 106.5 μs. FIG. 4D is a graph of Iᵥ in function of the number of pulses in a pulse train for trains at 80 Hz with a pulse duration of 106.5 μs.

FIGS. 5A, 5B and 5C illustrate activation maps for stimulation pulses with different stimulation current intensities, and with a pulse duration of 100 μs each. FIGS. 5A1, 5B1 and 5C1 show projections of the activated fibres onto the visual field corresponding to the activation maps of FIGS. 5A, 5B and 5C respectively. FIG. 5D illustrates the proportion P of fibres activated at a given stimulation current intensity for fibres of different diameters. FIG. 5E illustrates the co-ordinate system used.

FIG. 6A is a graph showing perceived luminosity L in function of stimulus strength S. FIG. 6B is a graph showing means and standard deviation of phosphene eccentricity w in function of stimulus strength S. FIG. 6C is a graph illustrating phosphene area: the probability (in %) that phosphenes have a certain disk radius p, in function of stimulus strength. FIG. 6D illustrates a comparison between experimentally obtained data and model predictions of phosphenes calculated by means of mathematical functions.

### DESCRIPTION OF THE ILLUSTRATIVE EMBODIMENTS

The present invention will be described with reference to certain embodiments and drawings but the present invention is not limited thereto but only by the claims.

As an example, vision rehabilitation for a human person or animal according to the present invention may be done by electrically stimulating a cuff electrode which encircles the optic nerve as described in WO 00/06248. However, the present invention is not limited thereto. The cuff electrode may have a plurality of contacts, e.g. four contacts installed around the optic nerve, one at 0°, one at 90°, one at 180° and one at 270°. WO 00/06248 describes methods of extracting phosphene data from a patient, electrodes and their positioning and pulse shapes and pulse trains for stimulating phosphenes. WO 00/06248 is incorporated herein by reference.

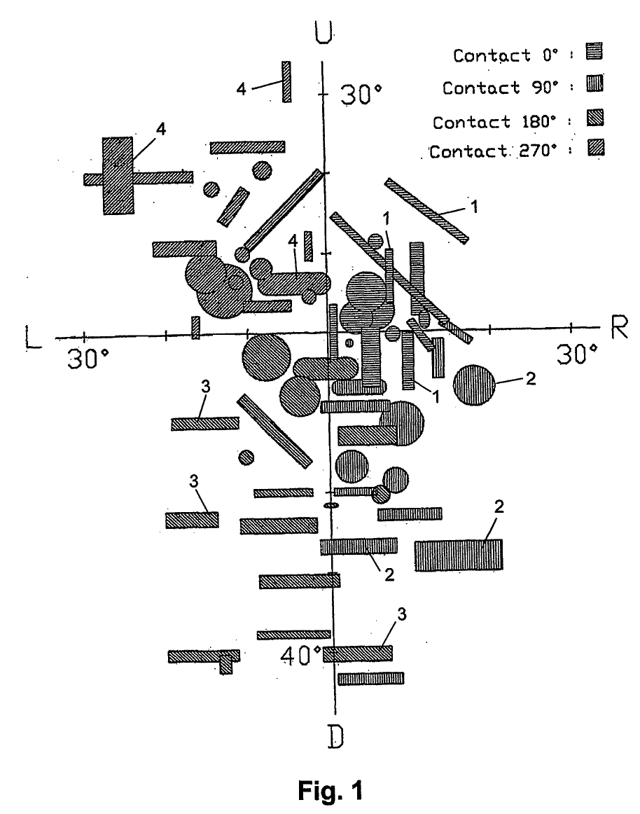

FIG. 1 shows a schematic representation of the arrangement of phosphenes perceived by a person according to activated, contacts in the spiral cuff electrode around the optic nerve. Different phosphenes are generated for different stimuli, provided that a stimulation threshold has been reached. Stimulating a first electrode contact generates phosphenes 1, stimulating a second electrode contact generates phosphenes 2, stimulating a third electrode contact generates phosphenes 3, and stimulating a fourth electrode contact generates phosphenes 4. The sizes, shapes and orientations of the phosphenes differ from each other. Although shown in black-and-white, phosphenes can be coloured.

Such a representation of generated phosphenes in function of different stimuli may be represented and stored in any suitable form in a digital phosphene data file, for example, a look-up table. In case of a look-up table, it preferably comprises both the stimulus which leads to a phosphene, as well as a description of the phosphene itself (comprising e.g. midpoint position, size, optionally orientation and/or shape). FIG. 1 only shows phosphenes generated by stimulating one of the contacts at a time, but of course the representation of generated phosphenes may also comprise phosphenes generated following the simultaneous stimulation of two or more contacts. Thus, FIG. 1 shows only a subset of the phosphenes which can be used with the present invention.

Phosphenes generated following an applied stimulus, are very personal. Therefore, an individual phosphene data file is preferably made for each person making use of a method and/or device according to the present invention.

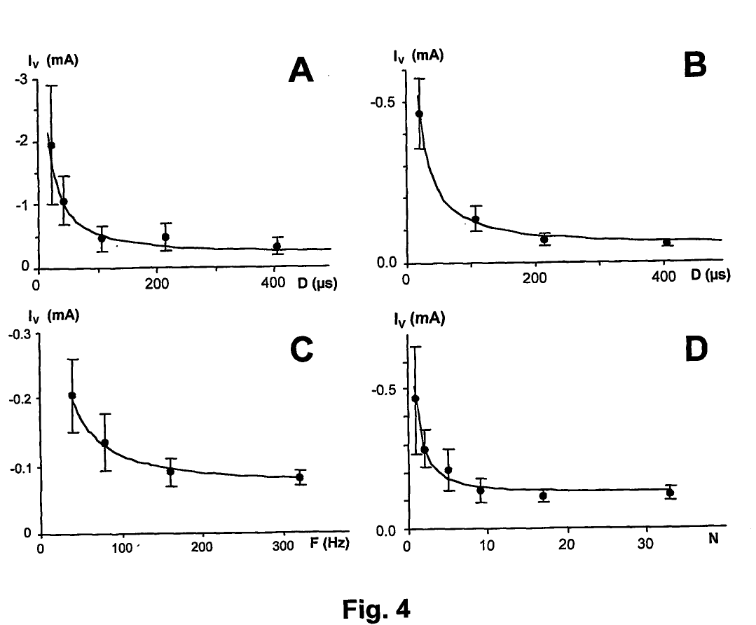

The phosphene perception threshold Iᵥ, which is the level of stimulation at which a transition occurs from a state of no perception to a state of seeing a phosphene, measured with a two staircase method as described in J. Delbeke et al., Invest Ophthalmol Vis Sci 42, 291 (2001) is much reduced by lengthening the stimulation pulse duration. This is illustrated in FIG. 4, showing visual perception thresholds Iᵥ in function of stimulus parameters. Dots represent average thresholds, and standard deviations are given as error bars. Models are plotted as continuous traces.

The classical Hill equation can be fitted to thresholds obtained by a single pulse, as represented in FIG. 4A, as well as to thresholds obtained by train stimulation, as represented in FIG. 4B. FIGS. 4A and 4B are graphs of perception threshold Iᵥ in function of stimulus duration D. FIG. 4A is a graph of 70 measurements with single pulse stimulation, FIG. 4B is a graph of 25 measurements with 9 pulse trains at 80 Hz. A same time constant c of 188 μs applies to both sets of data, but the rheobase (−0.21 mA for single pulses in FIG. 4A and −0.052 mA for the 9 pulse trains at 80 Hz in FIG. 4B) differs. The rheobase Iᵣ indicates the pulse amplitude at which phosphene perception will not be improved by an increase in pulse width. The rheobase Iᵣ is a function of the number N of pulses and their frequency F so that the strength-duration equation can be written as in equation (1).

$${I}_{V} = \frac{{I}_{r} ( N , F )}{1 - {ⅇ}^{- D / c}} \qquad ( 1 )$$

The strength-duration equation or curve reflects how much pulse amplitude and pulse width are necessary to generate a phosphene. This curve demonstrates how pulse amplitude and pulse width interact. At a point on the graph the curve flattens out to show the rheobase or the point where progressive increases in pulse width no longer affect the pulse amplitude.

A marked reduction of the perception threshold was observed when either frequency or number of pulses in the stimulation signal increased, as illustrated in FIG. 4C and FIG. 4D. FIG. 4C is a graph of 38 measurements of perception threshold Iᵥ in function of the pulse train frequency F for trains of 9 pulses with a duration of 106.5 μs, and FIG. 4D is a graph of 92 measurements of perception threshold Iᵥ in function of the number N of pulses in a pulse train for trains at 80 Hz with a pulse duration of 106.5 μs. The model traces in FIG. 4C and FIG. 4D correspond to equation (8) (see further), with the same time constant c (188 μs) as in FIG. 4A and FIG. 4B. The changes observed reach far beyond the well known ‘supernormal’ membrane excitability observed on repetitive stimulation as explained in E. D. Adrian, K. Lucas, J Physiol (Lond) 68 (1912). Some central integration mechanism has therefore been hypothesised whereby perception thresholds can be reached either through spatial summation, each single pulse recruiting more axons, or by temporal summation of the repeated activation of a few axons in case of train stimulation.

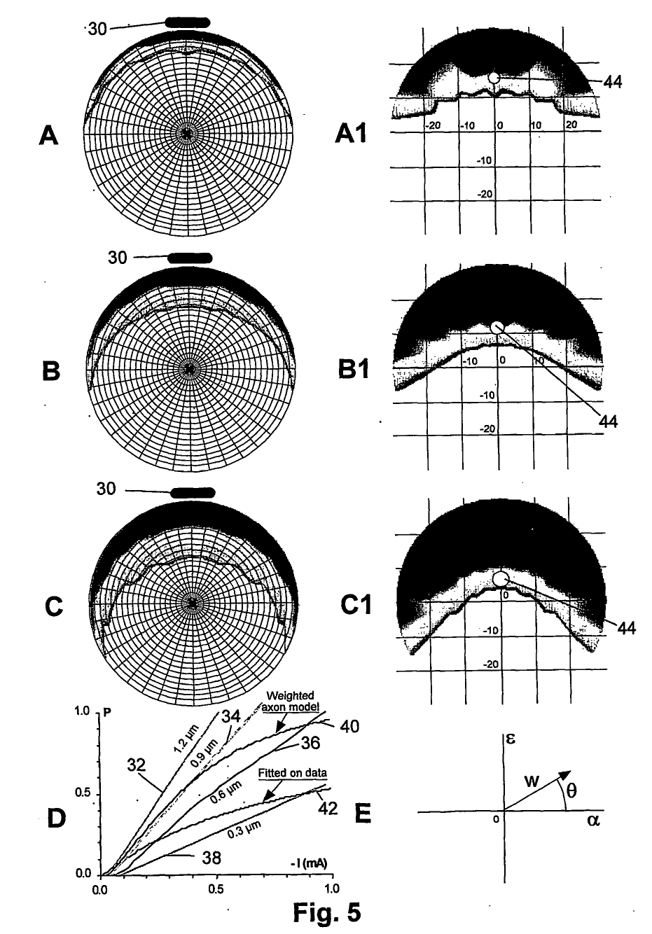

Spatial summation is directly dependent on the recruitment of fibres in the optic nerve section. A finite element geometry and a model of the optic nerve fibres may be used to map the expected distribution of fibres activated in a given stimulating condition, as shown in FIGS. 5A, 5B and 5C. FIGS. 5A, 5B and 5C show activation maps for stimulation pulses of 0.10 mA, 0.15 mA and 0.20 mA respectively, with a pulse duration of 100 μs. It is to be noted that these activation maps should only be interpreted qualitatively. As FIGS. 5A, 5B and 5C show, increasing the current results in an increased area or proportion of the nerve section where fibres of a given diameter are activated. The bars 30 in FIGS. 5A, 5B and 5C indicate stimulation contact positions. Different grey values in FIGS. 5A, 5B and 5C refer to a given optic nerve axon diameter.

Phosphene descriptions or representations may be conveniently expressed in azimuth (α) and elevation (ε) co-ordinates of the visual field. As illustrated in FIG. 5E, these have been transformed into eccentricity w and position angle θ. Activation maps are projected onto the visual field, as shown in FIGS. 5A1, 5B1 and 5C1, limited to 60 degrees. These projections take into account a central magnification factor defined as follows

x=1.685(1−e^{w/3.8})

where w is the eccentricity in the optic nerve expressed in mm. The circular regions 44 superimposed on these maps give a qualitative representation of the expected phosphenes for train stimuli of five pulses of the same current intensity delivered at 100 Hz.

FIG. 5D illustrates the proportion P of fibres activated at a given stimulation current intensity. The ratio P of this activated area to the full section area is taken as an estimate of the recruitment curve. The straight lines 32, 34, 36, 38 in FIG. 5D correspond to the modelled recruitment of fibres of a single diameter. The recruitment P was simulated for a nerve fibre population of a single diameter of 0.6 μm. The resulting graph 36 can be closely matched by a straight line. Additional testing for fibres of 0.3 μm, 0.9 μm and 1.2 μm diameter provided a set of such lines 32, 34, 38 differing by their slope and point of onset. The graph 36 of the 0.6 μm fibre has been fully modelled, while the three other line graphs 32, 34, 38 are extrapolated from a few points only. The following extrapolation function could be calculated:

P(d)=I(1.6d+0.142)−0.066

I being the stimulation current intensity in mA, and d being the fibre diameter in μm.

Taking into account the relative number of axons at 0.1 μm diameter bin width from 0.25 μm to 2.95 μm plus a group value around 5 μm, a weighted recruitment curve 40 is obtained that represents the proportion of activated fibres P including all diameters. The shape of this global recruitment curve could be fitted with the following empirical equation:

$$P = \frac{{I}_{r} - g}{{I}_{r} + h - 2 \cdot g} for {I}_{r} \geq g \qquad ( 2 )$$

where g is the rheobase current for activation of the first optic nerve axon recruited and h the current level at which half the axon population is activated with a single pulse of infinite length.

When values of g and h are fitted to the experimental thresholds as explained further, equation (2) does reveal a much slower recruitment than expected from the weighted recruitment curve as can be seen from graph 42 in FIG. 5D.

Because of the “all or none” behaviour of excitable membranes, equation (2) is applicable to each pulse within a train. If P_{N} represents the value of P at visual perception threshold for a train of N pulses, then, by rearranging equation (2) and substituting in equation (1),

$${I}_{V} = \frac{g + {P}_{N} \cdot ( h - 2 g )}{( 1 - {ⅇ}^{- D / c} ) \cdot ( 1 - {P}_{N} )} \qquad ( 3 )$$

Considering spatial summation, n is defined as the total number of fibres potentially involved in phosphene perception, P₁ is defined as the proportion of axons activated at perception threshold by a single pulse, M_{N} is defined as the post-synaptic membrane potential of an hypothetical integrating neuron after stimulation with a train of N pulses, and a is defined as the average amplitude contribution of each excitatory postsynaptic potential (EPSP). Then, at perception threshold,

M₁=n.P₁.a+M₀+M_{c}

where M₀ represents the residual potential generated by previous activity and M_{c} the influence of interfering factors such as e.g. other synchronous stimuli delivered, or background activity. Defining P_{S} as the value of P₁ in the absence of such interfering activity and for an isolated initial pulse, then the membrane potential is given by

M_{S}=n.P_{S}.a (4)

Let each EPSP be roughly represented by a decreasing exponential with time constant τ and an initial instantaneous amplitude a. Then, at perception threshold after a second pulse in a train of frequency F:

$${M}_{2} = n \cdot {P}_{2} \cdot a \cdot ( {ⅇ}^{- \frac{1}{τ F}} + 1 ) + {M}_{0} \cdot {ⅇ}^{- \frac{1}{τ F}} + {M}_{c}$$

By recurrence, the membrane potential for perception threshold at the onset of the last pulse of a train of N pulses at frequency F can be written as:

$${M}_{N} = n \cdot {P}_{N} \cdot a \cdot ( {\sum }_{i = 1}^{N} {ⅇ}^{\frac{i - N}{τ F}} ) + {M}_{0} \cdot {ⅇ}^{- \frac{N - 1}{τ F}} + {M}_{c} \qquad ( 5 )$$

Considering that at perception threshold, M_{N}=M_{S}, then the combination of equations (4) and (5) yields

$${P}_{N} = \frac{{P}_{s} \frac{1}{n \cdot a} ( {M}_{c} + {M}_{0} \cdot {ⅇ}^{- \frac{N - 1}{τ F}} )}{{\sum }_{i = 1}^{N} {ⅇ}^{\frac{i - N}{τ F}}} \qquad ( 6 )$$

For isolated trains, M₀=0 and M_{c}=0. Then

$$\frac{{P}_{S}}{{P}_{N}} = {\sum }_{i = 1}^{N} {ⅇ}^{\frac{i - N}{τ F}} . \qquad ( 7 )$$

Equation (7) can be substituted in equation (3) to obtain the expected perception threshold:

$${I}_{V} = \frac{g \cdot {\sum }_{i = 1}^{N} {ⅇ}^{\frac{i - N}{τ F}} + {P}_{S} \cdot ( h - 2 g )}{( 1 - {ⅇ}^{- D / c} ) \cdot ( {\sum }_{i = 1}^{N} {ⅇ}^{\frac{i - N}{τ F}} - {P}_{S} )} \qquad ( 8 )$$

The adequacy of this perception threshold model can be appreciated in the examples of FIGS. 4C and 4D.

The various parameters defined above have been estimated by fitting the threshold model to all threshold measurements (experimental data) available. Table I summarises these estimations for 215 stimulations, 59 at each of four contacts installed around the optic nerve.

TABLE I

| Iᵥ | c (μs) | τ (s) | g (μA) | h (μA) | Pₛ (0 . . . 1) | Number |
|---|---|---|---|---|---|---|
| 0° | 244 | 0.056 | 23.4 | 1906 | 0.059 | 59 |
| 90° | 219 | 0.066 | 20.3 | 2188 | 0.059 | 55 |
| 180° | 137 | 0.091 | 8.4 | 2405 | 0.107 | 52 |
| 270° | 150 | 0.072 | 7.5 | 2224 | 0.108 | 49 |
| Average | 188 | 0.071 | 14.9 | 2181 | 0.083 | 215 |

A determination coefficient r² of 0.81 [t(213)=30; p<0.001] is obtained between the experimental thresholds and the model results. The distribution of the threshold deviation from the expected values is roughly Gaussian with a standard deviation of 36%.

With a pulse intensity I above threshold, the stimulus strength S is defined as:

$$S ≡ ( \frac{I - {I}_{V}}{{I}_{V}} ) \cdot 100 \%$$

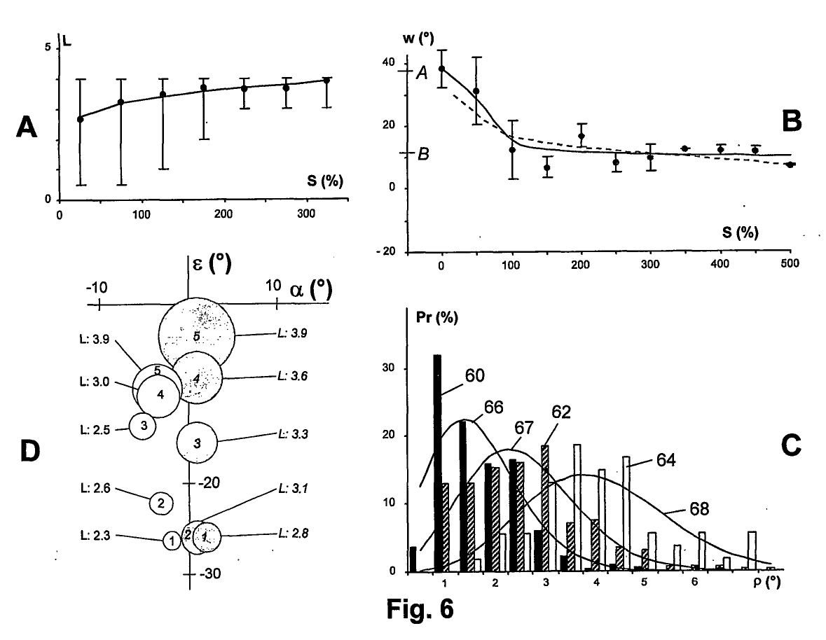

Phosphene luminosity, size and position have been explored using correlation with S as well as pulse duration D, number N and frequency F in order to identify the best predictor. These results are summarised in FIG. 6.

A luminosity grading scale from 0 (“no perceptions”) to 9 (“extremely bright”) was defined with a volunteer, but 98.9% of the levels ranged from 0 to 4 (“average”). The corresponding 1413 recordings are illustrated in FIG. 6A where average luminosity values obtained at different stimulus strengths S are plotted along with the corresponding ranges. Dots represent average values, while error bars indicate the full range of luminosity reported at the corresponding strength level. The perceived luminosity L has been found to correlate very significantly [r²=0.18; t(1409)=17.66; p<0.001] with the square root of the stimulus strength S according to

L=2.76+0.066·\sqrt{S} (9)

The line trace in FIG. 6A shows the expected values according to equation (9). Neither D, nor N nor F account for more than 1% of the luminosity variance. No significant difference could be detected between the results obtained from stimulation through each of the four contacts.

A phosphene ‘position’ refers to the barycenter of the pixels used to represent it: the phosphene position coordinates are the average value of the corresponding co-ordinates of all the pixels representing the phosphene. At threshold, phosphenes are typically located in an eccentric position defined as A. When the stimulus strength S is increased, the eccentricity reduces and phosphenes ultimately stabilise at a central position defined as B, for S above 200% as can be seen in FIG. 6B. This migration phenomenon is reminiscent of the stimulus intensity dependent shift of the midpoint of the margin of the activation maps, drawn as a dotted line in FIG. 6B.

B co-ordinates are estimated by averaging the 54 experimental values with stimulus strength S≧200%. A, however, is variable and dependent on the stimulus parameters. Except for contact 270°, where migration is hardly perceptible, the best predictor for A appears to be the ratio P_{S}/P_{N} as defined in equation (7) with the following linear regression

$$A = v + u \cdot {\sum }_{i = 1}^{N} {ⅇ}^{\frac{i - N}{τ F}} \qquad ( 10 )$$

Equation (10) applies to w and θ co-ordinates, the corresponding values of v and u being estimated from 355 experimental data obtained at S<30%.

In analogy with phosphene migration and the shift of the recruitment curve margin (FIGS. 5A1, 5B1, 5C1 and FIG. 6B), the following function was built:

$$A = v + u \cdot {\sum }_{i = 1}^{N} {ⅇ}^{\frac{i - N}{τ F}} \qquad ( 10 )$$

with Z=A if S<s where Z represents either w or θ depending on which set of constants is used. Constants s and m are the S values at onset of migration and at half way between A and B respectively. Equation (11) was fitted to the 256 results of migration experiments with S values between 30 and 200. Table II provides the equations (10) and (11) constants related to each contact. The significance of the correlation between the experimental data and the model are given as p values.

TABLE II

| | u_{w} (°) | v_{w} (°) | B_{w} (°) | s_{w} (%) | m_{w} (%) | P |
|---|---|---|---|---|---|---|
| 0° | 2.40 | 6.37 | 2.73 | 5.07 | 14.82 | <0.001 |
| 90° | 3.68 | 5.80 | 9.06 | 40.22 | 54.00 | <0.001 |
| 180° | 3.05 | 7.81 | 1.15 | 32.47 | 107.61 | <0.001 |
| 270° | −0.33 | 12.32 | 5.75 | 0 | 999999 | NS |
| | u_{θ} (°) | v_{θ} (°) | B_{θ} (°) | s_{θ} (%) | m_{θ} (%) | P |
| 0° | −3.55 | −36.35 | 13.98 | 0 | 56.14 | <0.001 |
| 90° | −3.13 | −54.74 | −84.06 | 0 | 0.69 | NS |
| 180° | −3.43 | −67.48 | −145.10 | −1 | 0 | NS |
| 270° | −6.23 | −153.40 | −224.96 | 85.72 | 123.13 | <0.001 |

Globally, the model yields an r² value of 0.54 [t(663)=27.7; p<0.001] and 0.50 [t(663)=25.5; p<0.001] for w and θ respectively.

The phosphene area computed by the sum of pixels representing it is transformed into an area equivalent disk of radius p. FIG. 6C shows the distribution of ρ at three stimulation strengths (S≦30%— black bars 60 in FIG. 6C; 30≦S<200%-hatched bars 62; S≧200%—white bars 64). R is the average value of ρ in each S group. These distributions of the phosphene diameters closely match the Poisson equation

$$Z = A + ( B - A ) \cdot ( \frac{S - s}{S + m - 2 s} ) \qquad ( 11 )$$

with 2R respectively equal to 3.22°, 4.45° and 6.91°. These values can thus be considered as estimates of the averages (2.96°, 4.51°, 6.15°) as well as of the variances (2.10°, 4.45°, 7.18°) of the equivalent diameters. The superimposed traces 66, 67, 68 in FIG. 6C show Poisson distributions fitted to the equivalent diameter distributions.

A determination coefficient r²=0.34 [t(663)=18.6; p<0.001] is obtained with following equation

ρ=1.63+0.012S (12)

D, N or F are each responsible for less than 5% of the variability of p as estimated from r².

The phosphene model of equations (9) to (12) was briefly assessed. The results are illustrated in FIG. 6D. Position, size and luminosity (L labels) are provided for both modelling predictions (grey disks) and experimental results (white disks) for different S values as indicated within each disk (1: stimulus strength 0%, 2: stimulus strength 25%, 3: stimulus strength 75%, 4: stimulus strength 150%, 5: stimulus strength 300%). In both cases (model and experiment), the stimulation conditions were the same: contact 180°, train of 9 pulses of 42.6 μs at 160 Hz.

It has been shown thus that the size of a phosphene does not account for the large number nor the topography of the fibres activated in the optic nerve. The axons involved in the visual perception appear to be located near the margin between activated and inactive fibres in the nerve section. Both move similarly in response to stimulation intensity changes.

Moreover, for a given stimulating condition (pulse duration, current intensity, number of pulses and frequency) the average phosphene attributes (size, position and luminosity) can be fairly predicted. The ratio of pulse intensity to threshold is a good predictor for size and luminosity.

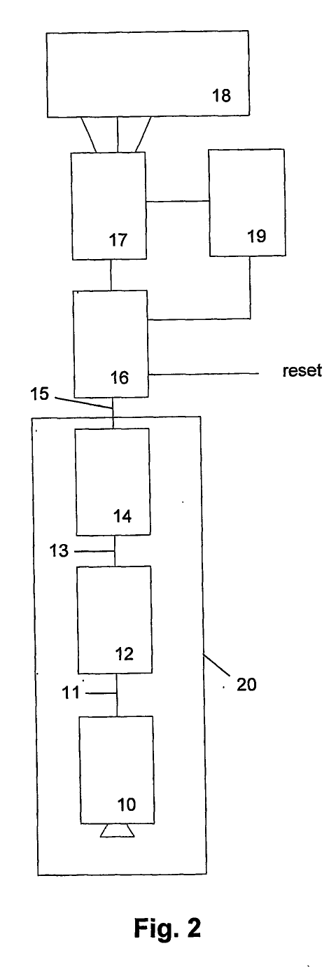

An image grabbing device such as e.g. a video camera 10 (FIG. 2) is used to capture an image. The output signal 11 of the video camera 10, representing the captured image, is converted in a digital format using an analog-to-digital converter 12. The obtained digital image 13 may be pre-processed in a pre-processor 14. Pre-processing may include, for example, one or more of resolution adaptation, thresholding, spatial and temporal filtering, region segmentation, selective spatial decimation (trough a mapping table), contrast enhancement, edge detection and gain modulation by local movement. Some aspects of image manipulation are described in “Traitement de I'Image sur Micro-ordinateur”, by Toumazet, Sybex, Paris, France, 1987. An additional global movement detection can also be provided. The pre-processed image 15 to be visualised is composed of an array of pixels. The pixel array may be of constant resolution or may have a spatially varying resolution, e.g. a log-polar map. The video camera 10, the A/D converter 12 and the pre-processor 14 together form means 20 for providing an image to be visualised by the visual system. The pre-processed image 15 is sent to an internal image memory 16.

At system initialisation, all pixels of the internal image memory 16 are set to zero. Next, a new pre-processed image frame 15 is added or integrated, pixel-to-pixel, to the internal image memory 16.

In accordance with the present invention the image to be visualised may be segmented into regions of similar light intensity and/or colour. Each region contains one or more pixels and a single value of intensity may be ascribed to all the pixels in the region. Various region segmentation schemes are known to the skilled person and may be used with the present invention. Ideally, a region-coding scheme should be chosen which optimally aligns region boundaries with functional elements of an image. As real images contain a large range of light intensities some thresholding to reduce the number of light intensities as well as some averaging of light intensities may be used to provide reasonably sized regions. Ideally, regions should be related to functional boundaries in the image—that is regions should not cross functional boundaries, such as from a face of a person to the background. The trade-off between region size and the variation in pixel intensities is well known to the skilled person of region coding. Below one method of segmentation is described based on selection of the brightest regions. A colour video camera may be used, even if the phosphene map is monochromatic as colour may be used to segment the image more effectively.

In a first step of an embodiment of the present invention, at least the largest, i.e. the brightest pixel or pixel region of the image stored in the internal image memory 16, is identified. Then, one or a number of most appropriate phosphenes to represent the brightest region is/are selected by a selector 17 from a look-up table 18. The selector may take into account at least the size and the location of the phosphene. That is, the selector compares the centre location and a size parameter (or actual size) of a phosphene with the brightest region and selects the phosphene if there is a match of at least the centre location to within a distance tolerance. The image 19 which would be generated by the selected phosphene(s) is then subtracted pixel-by-by pixel from the original image to form a current image in the image buffer. A new determination of the brightest pixel or pixel region is then made followed by a further selection of phosphene(s). In subsequent steps, this procedure is repeated until the image has been processed down to a certain threshold, or to when no phosphene can be matched with the remaining image. In this way, the pixel-based image is transferred into a summation of patches or regions of light whereby the available phosphene locations, shapes and sizes have been optimally used to match the phosphenes to these regions.

Instead of an iterative process as described above, a set of brightest regions of the image may be determined in one step, e.g. by a suitable block or region image segmentation algorithm. A limit of the regions may be set, e.g. regions below a certain number of pixels are ignored, and the decision algorithm may be made more complex to suit real-life situations, e.g. very bright regions may be accepted even if very small as they may represent vital image data. Hence, region segmentation may be done based on a variety of image parameters in order to extract suitable image regions.

As explained above, ‘most appropriate’ selection stands for different criteria that can be applied in different embodiments of the system. In one embodiment the selector selects the phosphenes of a look-up table characterised by a midpoint position and a size reference which fit best to the pixels of the image in the internal image memory 16 having the largest amplitude. The fit may be made using an appropriate algorithm, e.g. a least squares difference calculation between the location of pixels of the image and the pixels of the image which would be provided by the selected phosphenes. Actual phosphene size and orientation, and even phosphene shape, may also be taken into account.

When several phosphenes are suitable, the one not used for the longest period of time is preferably selected. The look-up table is preferably arranged in such a way that the most efficient stimuli (i.e. covering the largest pixels group), for example synchronous stimuli (single pulses or trains), are used first.

The phosphene selection above is repeated a number of times in order to obtain all the phosphenes that can be interlaced. Interlacing means here stimulation pulse trains, or synchronised pulse trains, interleaved with one another, with an appropriately adjusted delay.

The look-up table 18 also contains information on which contact or contacts need to be stimulated to generate the selected phosphenes, and what the corresponding stimuli parameters are (pulse duration, pulse repetition, pulse intensity). Once the phosphene selection is finished, the corresponding stimuli parameters are read out from the relevant look-up table 18 and sent to the signal generator to generate the electrical stimulation signals. The output of the signal generator are then transmitted to the appropriate contact(s) which is (are) then stimulated with the appropriate stimuli parameters, thus generating the phosphenes, and thus a phosphene generated image 19.

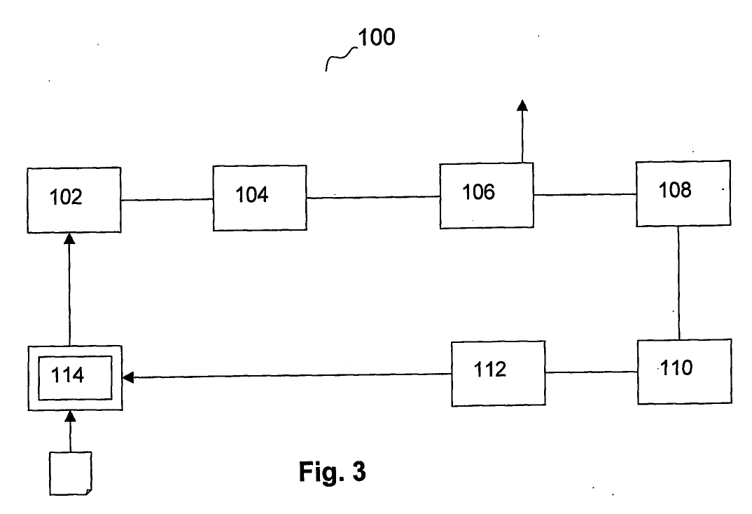

In one embodiment of the present invention real time images may be visualised by use of a leaky integrator and will be described with reference to the scheme 100 of FIG. 3.

In a first step 102, at least the largest, i.e. the brightest pixel or pixel regions of the image stored in the internal image memory 16, are identified. A suitable block or region segmentation algorithm may assist this step. In step 104, a number (1 or more) of appropriate phosphenes to represent the brightest regions are selected by a selector 17, e.g. from a look-up table 1. The selected phosphene descriptors are output in step 106. The selector may take into account at least the size and the location of the phosphenes in the selection step. The procedure is adapted for dynamic images by using a leaky integrator. The purpose of the leaky integrator is to allow the image stored in the image memory 16 to change with time but to filter out rapid or inconsequential changes of the image. So each image stored in the integrator decays with time. In step 108 the phosphene generated image 19, being image data corresponding to each phosphene selected, is subtracted pixel-by-pixel from the current image in the internal image memory 16 to form a new current image in step 110. For example, this generated image 19 multiplied by the corresponding brightness value as gain factor is returned pixel by pixel as a negative feedback to the input of the integrator so that the whole system behaves as a low-pass filter in time. In a more complete embodiment, timing correction factors could be added to the already mentioned intensity value.

Each further step in the cycle deals with the pre-processed image 15 working for each pixel as a leaking integrator. If implemented in a numerical form for example, the process cycle continues in step 112 with the division of the intensity of each pixel of the pre-processed image 15 by a selected factor (for example intensity division by a factor 2), representing the integrator leak. The leak or division factor must be large enough to keep the system stable, taking into account the frequency of the addition of new frames. For stability, all pixel values below a given threshold can also be set to zero thus suppressing low grey levels. Setting this threshold parameter to an elevated pixel value will set the system in a ‘no memory’ state if required for testing.

Thereafter, a new pre-processed image frame 15 is added or integrated, pixel-to-pixel, to the internal image memory 16 in step 114, and a new set of most appropriate phosphenes is selected by the selector 17 for image regions in steps 102 and 104 as described above.

After an appropriately adjusted waiting time, setting the shortest allowable image repetition rate, this stimulation process 100 is looped again.

In accordance with a further embodiments of the present invention large changes in parts of the captured image, e.g. through motion of the image grabbing device may be used to reset the phosphene selection procedure. In accordance with one embodiment, in parallel with the above procedures, an output from a global movement detector linked to the camera 10 can be used to reset all integrators or to set all pixel values of the image in the internal image memory 16 to zero whenever large shifts are detected in the input signal. In accordance with another embodiment the motion may be detected by the analysis of the captured image. Motion detection using analysis of subsequent frames of a video image is well known to the skilled person and this motion detection can be used to reset all integrators or reset all pixel values in the image. Motion detection may also be carried out on a block-wise representation of the image. In this procedure the image in the image memory 16 is segmented into blocks and motion detection is carried out in each block of a succession of image frames. When motion is detected in one block in a frame the pixel values for this block may be set to zero.

One embodiment of a method of phosphene selection will now be described.

The initial captured digital image is divided into a plurality of blocks, e.g. 4×4 pixel blocks. For each block an error value is calculated. The error function E_{B} of a block may be defined by equation (13):

E_{B}=ΣF₁(pixel intensity),−summed over the pixels of a block. (13)

Any pixel which is activated (bright) is an error. The function F₁ may be any suitable function, either linear or non-linear. It may be selected to emphasize pixels with higher intensity, e.g. (pixel intensity)². After the error functions for all blocks have been calculated the block with the highest error value is selected (highest brightness). Based on the centre location of the block and its intensity value, a phosphene is selected which lies within the block. The image which would be generated by this phosphene taking into account its size is then subtracted from the relevant block. After this procedure the error functions for relevant blocks are recalculated (as only one block has been treated, only the error function for this block needs to be calculated). Again the brightest block is selected based on the error function calculation and a further phosphene selected for this block. The image which would be produced by this selected phosphene is then subtracted from this block, pixel-by-pixel. By repeating this procedure, the current image is reduced in intensity—effectively important parts of the image (as determined by pixel intensity) are removed from a current image to provide a further image which is a better adaptation of the current image to the image to be visualised. Ideally, after the complete process, only a black image should remain (all intensity has been eliminated by subtraction of selected phosphene images), however, due to the lack of perfect coverage of the phosphenes this will rarely be the case. After the process is complete or after a certain time as determined by the frame rate, stimulation signals for the selected phosphenes are generated and output. As indicated above, where there is a choice of several phosphenes, a phosphene which has not been used recently is preferably selected.

The remaining current image is then multiplied by a factor (to create a decay in these remaining values) and a new image is added to the modified current image. The complete process is then repeated.

In order to provide a better estimate of the error function, the image which would be created by a phosphene is not only subtracted from the selected block but also for any neighbouring block affected by the size of the phosphene. After this subtraction process, the error function for more blocks must be calculated (as more have been affected).

In yet a further embodiment, where a phosphene is bright when it should be dark for the best portrayal of the image, this false brightness is indicated in the relevant block of the current image as a negative intensity. The error function for such a block may be designed so that it takes into account not only the image to be portrayed and phosphenes which do not extend far enough to represent bright pixels (hence leaving bright areas in the current image which have not been eliminated) but also phosphenes which extend too far and introduce brightness in the image where there should not be any. To make use of this added feature, the sum of error functions for the complete image may be determined after each phosphene subtraction step. If the sum of all error functions does not reduce after the subtraction step (indicating that phosphene introduces more errors than it solves) the phosphene may be rejected and another phosphene sought.

The present invention includes means for carrying out any of the above method steps. Generally, these means will be implemented in software running on a suitable processor such as an Intel Pentium III processor running on a portable machine such as a lap top having the necessary non-volatile memory, random access memory, bus system and input/output ports.

While the invention has been shown and described with reference to preferred embodiments, it will be understood by those skilled in the art that various changes or modifications in form and detail may be made without departing from the scope and spirit of this invention. For example, the present invention also includes the use of any of the above apparatus or methods of the invention with a suitable electrode or electrode array for providing a visualisation rehabilitation device or method. The present invention also includes such a device as implemented on an animal, especially a human. The present invention also includes a method of treatment of a person or animal suffering from blindness or impaired vision by implanting a suitable electrode or electrode array on or in the visual system of the person or animal and driving the electrode or electrode array with signals generated in accordance with the above methods or supplied by the above apparatus in accordance with the invention. Transfer of the signals from the signal generator to the electrode or electrode array can be made by any suitable method, e.g. cable or wireless transmission.

1. A method for generating stimulation signals suitable for visualising an image by electrical stimulation of functional parts of a visual system of an animal, comprising:
a phosphene data file providing step for providing a record of a location of each phosphene of a plurality of phosphenes in the visual system, at least one phosphene having a different location than other phosphenes,
a selection step for selecting out of the phosphene data file a phosphene for visualising a region of the image; and
a stimulation signal generating step for generating stimulation signals for stimulating functional parts of the visual system so as to generate the selected phosphenes, at least one stimulation signal being suitable for changing the location of a phosphene in the visual system.

2. A method according to claim 1, wherein the stimulation signal generating step is such that at least some stimulation signals are suitable for changing the location of phosphenes so as to form an array of phosphenes.

3. A method according to claim 2, wherein the stimulation signal generating step is such that at least some stimulation signals are suitable for changing the location of phosphenes so as to form a regular array of phosphenes.

4. A method according to any of claims 2 or 3, wherein the image is provided in the form of pixels with a first resolution, the selected phosphenes each having a centre, wherein the phosphene centres are spaced on the array with a second resolution which allows one pixel of the image to correspond with a phophene.

5. A method according to any of the previous claims, the stimulation signal having an intensity, wherein the location of a phosphene is changed by adjusting the intensity of the stimulation signal.

6. A method according to any of the previous claims, the stimulation signal having a number of pulses in a pulse train, wherein the location of a phosphene is changed by adjusting the number of pulses in the stimulation signal pulse train.

7. A method according to any of the previous claims, the stimulation signal having a frequency, wherein the location of a phosphene is changed by adjusting the frequency of the stimulation signal.

8. A method for generating stimulation signals suitable for visualising an image by electrical stimulation of functional parts of a visual system of an animal, comprising:
a phosphene data file providing step for providing a record of a location and a size parameter of each phosphene of a plurality of phosphenes, at least one phosphene having a different size than other phosphenes,
a selection step for selecting out of the phosphene data file a phosphene for visualising a region of the image; and
a stimulation signal generating step for generating stimulation signals for stimulating functional parts of the visual system so as to generate the selected phosphenes, the generating step using at least a size parameter of a selected phosphene.

9. A method according to any of the previous claims, the image being provided in the form of pixels each having a brightness, wherein the selection step selects the phosphenes based on midpoint positions and a size parameter of phosphenes fitting the pixels of the image having the largest brightness.

10. A method according to claim 9, wherein the selection step further takes into account phosphene size and/or orientation.

11. A method according to any of the previous claims, wherein in the selection step two or more phosphenes fit an image region, further comprising the step of selecting phosphenes which have not been used for a predetermined period of time.

12. A method according to any of the previous claims, wherein the selection step selects addresses from look-up tables comprising phosphenes and their corresponding stimulation parameters to be used during the stimulation signal generation step.

13. A method according to any of the previous claims, wherein the image is provided by capturing an image with an image grabbing device.

14. A method according to any of the previous claims, whereby synchronous stimulations, interleaved pulse trains and/or successive stimuli are used in the stimulation signal generating step to optimise information transmission to the visual system.

15. A method according to claim 14, furthermore comprising a step of integrating the image to be visualised.

16. A method according to claim 14 or 15, wherein a time sequence of images is provided and the images are integrated in a leaky integrator.

17. A method according to any of the previous claims, furthermore comprising a feedback step wherein image data of the image which would be created by the selected phosphenes is fed back as a negative feedback to a representation of the image to be visualised.

18. A method according to any of the previous claims, whereby fixed image extinction in the visual system is reduced through the sequential use of a succession of different phosphenes to represent a same constant light spot.

19. A method according to any of the previous claims, whereby the functional part of the visual system is any of the visual cortex, the retina, or the optic nerve.

20. A device for generating stimulation signals suitable for visualising an image by electrical stimulation of functional parts of a visual system of an animal comprising:
means for receiving an image to be visualised by the visual system,
a phosphene data store for storing at least a location of each phosphene of a plurality of phosphenes in the visual system, at least one phosphene having a different location than other phosphenes,
a selector for selecting out of the phosphene data store a phosphene for visualising a region of the image, the selector using the location parameter of a selected phosphene, and
a signal generator for generating stimulation signals for stimulating the functional parts of the visual system so as to generate the selected phosphenes, at least one stimulation signal being suitable for changing the location of a phosphene in the visual system.

21. A device according to claim 20, wherein the signal generator comprises means for generating at least some stimulation signals suitable for changing the location of phosphenes so as to form an array.

22. A device according to claim 21, wherein the signal generator comprises means for generating at least some stimulation signals suitable for changing the location of phosphenes so as to form a regular array.

23. A device according to any of claims 20 to 22, the stimulation signals having an intensity, wherein the signal generator comprises means for adjusting the intensity in order to change phosphene location.

24. A device according to any of claims 20 to 23, the stimulation signals having a number of pulses in a pulse train, wherein the signal generator comprises means for adjusting the number of pulses in a stimulation signal pulse train in order to change phosphene location.

25. A device according to any of claims 20 to 24, the stimulation signals having a frequency, wherein the signal generator comprises means for adjusting the frequency in order to change phosphene location.

26. A device for generating stimulation signals suitable for visualising an image by electrical stimulation of functional parts of a visual system of an animal comprising:
means for receiving an image to be visualised by the visual system,
a phosphene data store for storing at least a size parameter and a location of each phosphene of a plurality of phosphenes, at least one phosphene having a different size than other phosphenes,
a selector for selecting out of the phosphene data store a phosphene for visualising a region of the image, the selector using the size parameter of a selected phosphene, and
a signal generator for generating the stimulation signals for stimulating the functional parts of the visual system so as to generate the selected phosphenes.

27. A device according to any of claims 20 to 26, wherein the phosphene data store includes the size and orientation of at least some of the phosphenes.

28. A device according to any of claims 20 to 27, wherein the selector comprises a look-up table comprising references to phosphenes and their corresponding stimulation parameters.

29. A device according to any of claims 20 to 28, wherein the phosphene parameters are defined by equations and the selector comprises calculating means to calculate parameter equations describing the phosphenes.

30. A device according to any of claims 20 to 29, further comprising an image grabbing device for capturing an image.

31. A device according to any of claims 20 to 30, wherein the signal generator comprises means for generating synchronous stimulations, interleaved pulse trains and/or successive stimuli to the functional parts of the visual system.

32. A device according to any of the claims 20 to 31, furthermore comprising an integrator for integrating the image to be visualised.

33. A device according to claim 32, furthermore comprising negative feedback means for feeding back to the integrator visualised image data corresponding to the selected phosphenes.

34. A method for generating stimulation signals suitable for visualising an image by electrical stimulation of functional parts of a visual system of an animal, comprising
an image providing step for providing a first image to be visualised—a phosphene data file providing step for providing a record of a location of each phosphene of a plurality of phosphenes,
a selection step for selecting out of the phosphene data file a phosphene for visualising a region of the first image,
subtracting an image corresponding to the selected phosphene from the first image to form a second image, and
a stimulation signal generating step for generating stimulation signals for stimulating functional parts of the visual system suitable to generate the selected phosphene(s).

35. A method according to claim 34, the image being provided in the form of pixels each having a brightness, wherein the selection step selects the phosphenes based on midpoint positions of phosphenes fitting the pixels of the first image having the largest brightness.

36. A method according to claim 35, wherein the selection step further takes into account phosphene size and/or orientation.

37. A method according to any of the claims 34 to 36, wherein in the selection step two or more phosphenes fit an image region of the first image, further comprising the step of selecting phosphenes which have not been used for a period of time.

38. A method according to any of the claims 34 to 37, wherein the selection step selects addresses from look-up tables comprising phosphenes and their corresponding stimulation parameters to be used during the stimulation signal generating step.

39. A method according to any of the claims 34 to 38, wherein the first image is captured by an image grabbing device.

40. A method according to any of the claims 34 to 39, wherein synchronous stimulations, interleaved pulse trains and/or successive stimuli are used in the stimulation signal generating step to optimise information transmission to the visual system.

41. A method according to claim 40, furthermore comprising a further image providing step to provide a third image to be visualised and a step of integrating the second image with the third image.

42. A method according to claim 40 or 41, further comprising providing a time sequence of images and integrating the images of the sequence in a leaky integrator.

43. A method according to any of the claims 34 to 42, wherein fixed image extinction in the visual system is reduced through the sequential use of a succession of different phosphenes to represent a same constant light spot in the first image.

44. A method according to any of the claims 34 to 43, wherein the functional part of the visual system is any of the visual cortex, the retina, or the optic nerve.

45. A device for generating stimulation signals suitable for visualising an image by electrical stimulation of functional parts of a visual system of an animal, comprising:
means for receiving a first image to be visualised,
a phosphene data store for storing at least a location of each phosphene of a plurality of phosphenes,
a selector for selecting out of the phosphene data store a phosphene for visualising a region of the first image,
means for subtracting an image of the selected phosphene from the first image to form a second image, and
a stimulation signal generator for generating stimulation signals for stimulating the functional parts of the visual system suitable to generate the selected phosphene(s).

46. A device according to claim 45, wherein the phosphene data store includes the size and orientation of at least some of the phosphenes.

47. A device according to claim 45 or 46, wherein the selector comprises a look-up table comprising references to phosphenes and their corresponding stimulation parameters.

48. A device according to any of claims 45 to 47, wherein the phosphene parameters are defined by equations and the selector comprises calculating means to calculate parameter equations describing the phosphenes.

49. A device according to any of claims 45 to 48, wherein the means for providing a first image comprises an image grabbing device for capturing an image.

50. A device according to any of claims 45 to 49, wherein the signal generator comprises means for generating synchronous stimulations, interleaved pulse trains and/or successive stimuli to the functional parts of the visual system.

51. A device according to any of the claims 45 to 50, furthermore comprising an integrator for integrating the image to be visualised.

52. A method to obtain a phosphene data file comprising a record of at least a size parameter of each phosphene of a plurality of phosphenes generated by a stimulation signal, the method comprising correlating a phosphene area of each phosphene with an intensity of the stimulation signal.

53. A method according to claim 52, furthermore comprising correlating luminosity of each phosphene with the intensity of the stimulation signal.

54. A method according to any of claims 52 or 53, furthermore comprising a step of fitting model equations to experimentally obtained phosphene data, those model equations having as parameters one or more of the following: stimulus strength, current intensity, pulse duration, number of pulses in a stimulation pulse train, frequency of a stimulation pulse train.

55. A method according to any of claims 52 to 54, the stimulation signal having a number of pulses in a pulse train, a pulse train having a frequency, the method furthermore comprising calculating a maximum eccentricity range for a phosphene in function of a frequency and a number of pulses in a stimulation purse train.

56. A method according to claim 54 or 55, furthermore comprising adjusting current intensity and/or pulse duration for adjusting phosphene position.

57. A method according to claim 56, furthermore comprising minimising duration of the stimulation signal.

58. A computer program product for executing any of the methods as claimed in claims 1 to 19 or 34 to 44 when executed on a computing device associated with a device for generating stimulation signals suitable for visualising an image by electrical stimulation of functional parts of a visual system of an animal.

59. A machine readable data storage device storing the computer program product of claim 58.
